SEQUENCE LISTING (16 sequences)

SEQ ID NO: 1 - DNA, 22 bp, synthetic construct
  source 1..22: mol_type = other DNA
cagtggttttaccctatggtag

SEQ ID NO: 2 - DNA, 21 bp, synthetic construct
  source 1..21: mol_type = other DNA
tagcagcacagaaatattggc

SEQ ID NO: 3 - DNA, 22 bp, synthetic construct
  source 1..22: mol_type = other DNA
tggtgtgtggttctatgccagc

SEQ ID NO: 4 - DNA, 21 bp, synthetic construct
  source 1..21: mol_type = other DNA
agcctcaagccagaattcagg

SEQ ID NO: 5 - DNA, 20 bp, synthetic construct
  source 1..20: mol_type = other DNA
aaatattggcacagggaagc

SEQ ID NO: 6 - DNA, 19 bp, synthetic construct
  source 1..19: mol_type = other DNA
agcccctcctcggtagttt

SEQ ID NO: 7 - DNA, 20 bp, synthetic construct
  source 1..20: mol_type = other DNA
ctgggggaacctgtcctatg

SEQ ID NO: 8 - DNA, 21 bp, synthetic construct
  source 1..21: mol_type = other DNA
tcattctgaacgcgcatgaag

SEQ ID NO: 9 - DNA, 23 bp, synthetic construct
  source 1..23: mol_type = other DNA
gtgctcgcttcggcagcacatat

SEQ ID NO: 10 - DNA, 23 bp, synthetic construct
  source 1..23: mol_type = other DNA
aaaaatatggaacgcttcacgaa

SEQ ID NO: 11 - DNA, 23 bp, synthetic construct
  source 1..23: mol_type = other DNA
ccttgtacatcgattcctgggtt

SEQ ID NO: 12 - DNA, 21 bp, synthetic construct
  source 1..21: mol_type = other DNA
cctgggctgcattatctggtg

SEQ ID NO: 13 - DNA, 20 bp, synthetic construct
  source 1..20: mol_type = other DNA
agaactggacccggttacca

SEQ ID NO: 14 - DNA, 20 bp, synthetic construct
  source 1..20: mol_type = other DNA
cacttcgctgatacccctga

SEQ ID NO: 15 - DNA, 22 bp, synthetic construct
  source 1..22: mol_type = other DNA
gtgacgttgacatccgtaaaga

SEQ ID NO: 16 - DNA, 19 bp, synthetic construct
  source 1..19: mol_type = other DNA
gccggactcatcgtactcc

(19) United States
(12) Patent Application Publication
(10) Pub. No.: US 2026/0158049 A1
(43) Pub. Date: Jun. 11, 2026

(54) METHODS OF PREVENTING CONGENITAL HEART DEFECTS INDUCED BY MATERNAL DIABETES USING ACTIVATORS OF MITOCHONDRIAL FUSION

(71) Applicant: University of Maryland, Baltimore, Baltimore, MD (US)

(72) Inventor: Peixin YANG, Ellicott City, MD (US)

(73) Assignee: University of Maryland, Baltimore, Baltimore, MD (US)

(21) Appl. No.: 18/978,760

(22) Filed: Dec. 12, 2024

Related U.S. Application Data
(60) Provisional application No. 63/610,513, filed on Dec. 15, 2023.

(51) Int. Cl. A61K 31/7032; A61K 31/277; A61P 9/00

(52) U.S. Cl. CPC A61K 31/7032; A61K 31/277; A61P 9/00

(57) ABSTRACT

Methods for preventing congenital heart defects (CHDs) in embryos of diabetic females via administration of mitochondrial fusion activators, such as teriflunomide and echinacoside echinacoside, are provided.

### STATEMENT OF FEDERALLY SPONSORED RESEARCH AND DEVELOPMENT

This invention was made with government support under Grant Number HL134368 awarded by the National Institutes of Health. The government has certain rights in the invention.

### SEQUENCE LISTING

A sequence listing in electronic (XML file) format is filed with this application and incorporated herein by reference. The name of the XML file is “2024-1715A_Sequence_Listing-1715A.xml”; the file was created on Dec. 11, 2024; the size of the file is 15,243 bytes.

### BACKGROUND OF INVENTION

The US infant mortality rate, a basic measure of public health, is among the highest among developed countries, and birth defects are a major cause of infant death [1]. Congenital heart defects (CHDs) are the most common cause of infant death [2]. Furthermore, CHDs are the most prevalent birth defects, occurring in approximately 4-10 per 1000 live births [2]. Epidemiological studies in CHD prevention suggest a controversial effect of maternal folic acid supplementation [3,4], which is the only effective intervention to prevent neural tube defects, another type of potentially fatal birth defect. However, a mechanism-based means of preventing CHDs is still lacking.

Human epidemiological studies have also demonstrated that non-genetic factors are the major contributing factors to the occurrence of CHDs [2,5]. Among all non-genetic factors that cause CHDs, maternal diabetes is the major factor [2,6]. The rate of CHDs in infants born to mothers with diabetes is approximately four to six times higher than mothers without diabetes [6-8]. More than 60 million women of reproductive age worldwide have diabetes, and this number will likely double by 2030 due to the current global epidemic of obesity [9]. Even under the best clinical care, women with diabetes are still three to four times more likely to have a child with CHDs than women without diabetes [10].

Given these incidences and the lack of means to prevent CHDs, its occurrence is an unmet clinical need, and therefore uncovering the cellular and molecular events underlying its development will aid in the identification of effective preventions. The present invention is directed to these and other important goals.

### BRIEF SUMMARY OF INVENTION

As reported herein, the present inventor has found through the use of a non-genetic mouse model involving pregestational maternal diabetes that congenital heart defects (CHDs) occur due to activation of the transcription factor FoxO3a, which stimulates expression of miR-140 and miR-195 that, in turn, represses mitofusin 1 (Mfn1) and mitofusin 2 (Mfn2) expression, respectively. Treating this model with two activators of mitochondrial fusion, teriflunomide (TERI), a U.S. Food and Drug Administration-approved drug, and echinacoside (ECH), a natural compound, prevention of CHDs was achieved. This amelioration via activation of mitochondrial fusion correlated with re-expression of Mfn1 and Mfn2, improved mitochondrial dynamics and cell proliferation, and reduced apoptosis. Thus, pharmacological restoration of mitochondrial fusion may be an effective approach to reducing risk of CHDs resulting from diabetic pregnancy.

The present invention is based on these discoveries and generally encompasses therapeutic applications for mammalian embryos in mothers afflicted with diabetes.

In a first embodiment, the invention is directed to methods of preventing congenital heart defects (CHDs) in an embryo of a pregnant diabetic female subject. These methods comprise administering to a pregnant diabetic female subject and/or an embryo of a pregnant diabetic female subject a therapeutically-effective amount of at least one mitochondrial fusion activator. The mitochondrial fusion activator may be, but is not limited to, teriflunomide (TERI) or echinacoside (ECH).

In a second embodiment, the invention is directed to methods of restoring or augmenting mitochondrial fusion in an embryo of a pregnant diabetic female subject. These methods comprise administering to a pregnant diabetic female subject and/or an embryo of a pregnant diabetic female subject a therapeutically-effective amount of at least one mitochondrial fusion activator. The mitochondrial fusion activator may be, but is not limited to, teriflunomide (TERI) or echinacoside (ECH). Mitochondrial fusion may be restored, for example, in cardiomyocytes.

In a third embodiment, the invention is directed to methods of inducing or augmenting expression of mitofusin 1 (Mfn1) and/or mitofusin 2 (Mfn2) in an embryo of a pregnant diabetic female subject. These methods comprise administering to a pregnant diabetic female subject and/or an embryo of a pregnant diabetic female subject a therapeutically-effective amount of at least one mitochondrial fusion activator. The mitochondrial fusion activator may be, but is not limited to, teriflunomide (TERI) or echinacoside (ECH).

In relevant aspects and embodiments of the invention, the therapeutically-effective amount of the mitochondrial fusion activator is a dose of between 0.1 mg/kg and 1000 mg/kg.

In relevant aspects and embodiments of the invention, the therapeutically-effective amount of TERI is a dose of between 1 mg/kg and 30 mg/kg.

In relevant aspects and embodiments of the invention, the therapeutically-effective amount of ECH is a dose of between 1 mg/kg and 30 mg/kg.

In relevant aspects and embodiments of the invention, wherein the embryo is in the first or early second trimester.

In relevant aspects and embodiments of the invention, the mitochondrial fusion activator is administered to the amniotic cavity of the embryo. Non-limiting examples of specific means of administration include microinjection.

In relevant aspects and embodiments of the invention, the female subject has gestational diabetes.

In relevant aspects and embodiments of the invention, the female subject is a female mammal.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1. Mitochondrial fusion activators ameliorate maternal diabetes-induced CHDs. (A) and (J) Primary cardiomyocytes transfected with mitochondrial matrix-targeted photoactive GFP (mito-PAGFP) and percentage of mitochondria (>2 μm) per area in cells (n=5). (B) Protein levels in E12.5 hearts (n=3). (C) mito-PAGFP in primary cardiomyocytes. Red circles: photoactivated regions. (D) mito-PAGFP intensity (n=3). (E) Time to first fusion event (n=5). (F) and (I) mRNA levels in E12.5 hearts (n=3). (G) Heart vessels (upper) and sections (lower). (H) and (K) Numbers of embryos. AO, aorta; ECH, echinacoside; HLHS, hypoplastic left heart syndrome. LA/RA, left/right atrium; LV/RV, left/right ventricle; PA, pulmonary artery; PTA, persistent truncus arteriosus; TERI, teriflunomide. Data are presented as mean±standard deviation (SD) (A), (B), (D), (E), (F), (I), and (J). Student's t test (A) and (B). One-way ANOVA with Tukey's post-hoc test (D), (E), (F), (I), and (J). Chi-square test (H) and (K). *P<0.05,**P<0.01 and ***P<0.001.

FIG. 2. Mitochondrial fusion activators reduce CHDs induced by miR-195/140 transgenic overexpression. miRNA levels in primary cardiomyocytes (n=3) (A) and in E12.5 hearts (B) (n=3). (C) and (H) mRNA levels in E12.5 hearts (n=3). (D) Time-lapse images of mito-PAGFP in primary cardiomyocytes. (E) mito-PAGFP fluorescence intensity (n=3). (F) Time to first mitochondrial fusion event (n=5). (G) and (I) Numbers of E17.5 embryos. dTg, miR-195 and miR-140 double transgenic; WT, wild-type. Data are presented as mean±SD in (A), (B), (C), (E), (F), and (H). One-way ANOVA with Tukey's post-hoc test (A), (C), (E), (F), and (H). Student's t test (B). Chi-square test (G) and (I). *P<0.05, **P<0.01 and ***P<0.001.

FIG. 3. FoxO3a upregulates miR-140/195 and represses mitochondrial fusion. (A) FoxO3a immunostaining in primary cardiomyocytes and quantification of fluorescence intensity in nuclei (n=3). FoxO3a binding sites on the Mir195 (B) and Mir140 (C) promoters and promoter-driven luciferase reporter activity in H9C2 cells (n=3). (D) miRNA levels in E12.5 hearts (n=3). (E) E17.5 heart vessels (upper) and sections (lower). (F) Numbers of E17.5 embryos. (G) Representative images of primary cardiomyocytes. (H) Percentage of mitochondria (>2 μm) per area in cells (n=5). (I) and (J) Flow cytometry analysis and ratios of fluorescence intensity (red/green) in primary cardiomyocytes (n=3). (K) Protein levels (n=3). TSS, transcription start site; VSD, ventricular septum defect. Data are presented as mean±SD in (A), (B), (C), (D), (H), (J), and (K). Student's t test (A). One-way ANOVA with Tukey's post-hoc test (B), (C), (D), (H), (J), and (K). Chi-square test (F). *P<0.05, **P<0.01 and ***P<0.001.

FIG. 4. miR-195 deletion restores mitochondrial fusion. (A) Images of E17.5 heart vessels (upper) and sections (lower). (B) Numbers of E17.5 embryos. TUNEL-positive cells in E9.5 hearts (C) and the quantification (D) (n=3). p-H3-positive cells in E9.5 hearts (E) and the quantification (F) (n=3). (G) mito-PAGFP intensity in primary cardiomyocytes (n=3). (H) Time to first mitochondrial fusion event (n=9). (I) Representative images of primary cardiomyocytes and percentage of mitochondria (>2 μm) per area in cells (n=9). (J) Flow cytometry analysis and ratios of fluorescence intensity (red/green) in primary cardiomyocytes (n=3). OFT, outflow tract. Data are presented as mean±SD in (D), (F), (G), (H), (I), and (J). Chi-square test (B). One-way ANOVA with Tukey's post-hoc test (D), (F), (G), (H), (I), and (J). *P<0.05, **P<0.01 and ***P<0.001.

FIG. 5. Inhibition of miR-195 improves mitochondrial fusion by upregulating Mfn2. (A) Representative images of primary cardiomyocytes. (B) Percentage of mitochondria (>2 μm) per area in cells (n=9 or 5). (C) miR-195 binding site on Mfn2 mRNA. (D) Mfn2 mRNA level bound to biotin-labeled miR-195 (n=3). (E) miR-195 and Mfn2 mRNA in RNA co-immunoprecipitation (IP) in E12.5 hearts (n=3). (F) Mfn2 mRNA in primary cardiomyocytes (n=3). (G) Mfn2 mRNA and protein levels (n=3). Data are presented as mean±SD in (B), (D), (E), (F), and (G). One-way ANOVA with Tukey's post-hoc test (B), (F), and (G). Student's t test (D) and (E). *P<0.05, **P<0.01 and ***P<0.001.

FIG. 6. miR-140 deficiency restores Mfn1 expression and mitochondrial fusion. (A) The miR-140 binding site on Mfn1 mRNA. (B) Mfn1 mRNA bond to biotin-labeled miR-140 (n=3). (C) miR-140 and Mfn1 mRNA levels in RNA immunoprecipitation (n=3). (D) Mfn1 mRNA and protein levels (n=3). (E) Representative images of primary cardiomyocytes. (F) Percentage of mitochondria (>2 μm) per area in cells (n=4). (G) mito-PAGFP intensity in primary cardiomyocytes (n=3). (H) Time to first mitochondrial fusion event (n=9). (I) Images of E17.5 heart vessels (upper) and sections (lower). Data are presented as mean±SD in (B), (C), (D), (F), (G), and (H). Student's t test (B) and (C). One-way ANOVA with Tukey's post-hoc test in (D), (F), (G), and (H). Chi-square test (I). *P<0.05, **P<0.01 and ***P<0.001.

FIG. 7. Restoring Mfn1 expression abrogates the teratogenicity of maternal diabetes. (A) Representative images of primary cardiomyocytes and percentage of mitochondria (>2 μm) per area in cells (n=4). (B) Time-lapse images of mito-PAGFP in primary cardiomyocytes. (C) mito-PAGFP intensity in primary cardiomyocytes (n=3). (D) Time to first mitochondrial fusion event (n=5). (E) E17.5 heart vessels (upper) and sections (lower). (F) and (I) Numbers of E17.5 embryos. (G) Cleaved caspases-3 levels (n=3). (H) TUNEL-positive cells in E9.5 hearts and the quantification (n=3). TGA, transposition of the great arteries. Data are presented as mean±SD in (A), (C), (D), (G), and (H). One-way ANOVA with Tukey's post-hoc test (A), (C), (D), (G), and (H). Chi-square test (F) and (I). *P<0.05, **P<0.01 and ***P<0.001.

FIG. 8. Teriflunomide and echinacoside in embryonic hearts measured by HPLC-MS after i.p. injection. (A) Five embryonic day 13.5 (E13.5) hearts were pooled for measurement. 10% DMSO+90% corn oil served as the vehicle. The bar graph shows the mean content of teriflunomide (TERI) per heart. (B) Five E13.5 hearts were pooled for measurement. Water served as the vehicle. The bar graph shows the mean content of echinacoside (ECH) per heart.

FIG. 9. miR-140 deletion recovers cell proliferation and survival in the developing heart. (A) Cell proliferation in embryonic day 12.5 (E12.5) hearts was detected by p-H3 staining. Quantitative data of the percentage of p-H3-positive cells in E12.5 hearts are shown. (B) Cell apoptosis in E12.5 hearts was detected by a TUNEL assay. Apoptotic cells are labeled in red. The quantification of the percentage of apoptotic cells is indicated in the bar graph. (C) Protein abundance of cleaved caspases-3 in each group. The bar graph shows the quantitative data. Three embryos from three litters were analyzed for each group (n=3). Data are presented as mean±standard deviation (SD) (A), (B), and (C). One-way ANOVA with Tukey's post-hoc test was used to analyze the data (A), (B) and (C). *P<0.05, **P<0.01 and ***P<0.001.

FIG. 10. Transgenic overexpression of Mfn2 in the embryonic heart reduces congenital heart defect (CHD) incidences in diabetic pregnancy. Images of major vessels and H & E staining of embryonic day 17.5 (E17.5) heart sections in wide-type (WT) and Mfn2 transgenic (Mfn2-Tg) embryos from nondiabetic (ND) and diabetic mellitus (DM) dams. The bar graph indicates the numbers of normal and CHD embryos. AO, aorta; PA, pulmonary artery; LA, left atrium; RA, right atrium; LV, left ventricle; RV, right ventricle; VSD, ventricular septum defect. Chi-square test was used to analyze the data. *P<0.05, **P<0.01 and ***P<0.001.

### DETAILED DESCRIPTION OF THE INVENTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

As summarized above, the present invention is generally directed to methods for preventing the formation of congenital heart defects (CHD) in mammalian embryos of mothers afflicted with diabetes.

It is conventionally accepted that organ development is orchestrated from the cell nucleus and that the mitochondria simply follow along. However, a recent study demonstrated that mitochondria orchestrate developmental events of the mouse heart, and the disturbance of mitochondrial function contributes to CHD formation [16]. Mitochondrial dynamics are governed by fusion and fission events essential for proper heart development [16]. Mitochondria fuse via the function of mitofusin 1 (Mfn1) and mitofusin 2 (Mfn2). Deleting the Mfn1 and Mfn2 genes in early heart muscle cells results in severely underdeveloped hearts [16]. Furthermore, mouse embryonic stem cells missing Mfn2 and Opal (optic atrophy protein 1), a mitochondrial fusion facilitator, do not develop into beating cardiomyocytes [16]. Reduced mitochondrial fusion resulting from Mfn1 and Mfn2 deletion disrupts several signaling pathways implicated in CHDs [16]. This evidence suggests that altered mitochondrial dynamics drive cardiac dysmorphogenesis.

Impaired mitochondrial fusion leads to mitochondrial dysfunction and subsequently alters cardiac morphogenesis. Mitochondrial dysfunction is an evident cellular defect in cardiomyocytes exposed to maternal diabetes [17,24]. Intrinsic abnormalities are present in cardiomyocytes derived from inducible pluripotent stem cells of patients with CHDs that lack an underlying genetic cause [25], suggesting that cell development is a key factor in cardiac morphogenesis. These cellular organelle defects continue to persist after the establishment of CHDs and may contribute to sustainable cardiomyocyte dysfunction in CHD patients. The experimental data presented herein for the first time demonstrates that maternal diabetes increases two key miRNAs which impair mitochondrial fusion and enhance mitochondrial fragmentation in mouse embryonic cardiomyocytes in vitro and in vivo.

miRNAs are critically involved in virtually all aspects of cardiac development and disease [26]. miR-1 overexpression disrupts mouse embryonic heart development [27], and miR-133a overexpression in cardiomyocytes leads to decreased cell proliferation and the formation of cardiac septation defects [28]. The experimental data presented herein demonstrates that the upregulation of miR-140 and miR-195 mediates the teratogenicity of maternal diabetes leading to CHDs. During development, miR-140 is predominantly expressed in embryonic chondrocytes [29]. miR-140 induces cardiomyocyte apoptosis via the intrinsic mitochondrial pathway [14]. In contrast to miR-140, miR-195 is expressed early in the developing human heart [30]. miR-195 inhibits cell proliferation and induces apoptosis by repressing multiple pro-survival proteins [31,32]. Multiple lines of evidence suggest that miR-140 and miR-195 always work together. They both trigger mitochondrial dysfunction [14,31], participate in stem cell aging [31], and are elevated in adult heart diseases [33]. In agreement with the coherence between these two miRNAs, the experimental data presented herein shows that deleting the mir 140 gene or the mir 195 gene significantly ameliorated maternal diabetes-induced CHDs, and that overexpressing these two miRNAs in the heart mimicked maternal diabetes in inducing CHDs.

The transcription factor FoxO3a is activated by maternal diabetes [23]. FoxO3a upregulates miRNAs in cancer cells [34]. The experimental data presented herein shows that FoxO3a transcriptionally induces miR-140 and miR-195 expression and thus inhibits mitochondrial fusion in embryonic cardiomyocytes. FoxO3a reduces the sizes of cardiomyocytes in rats [35]. FoxO3a is a cell death trigger that acts through the mitochondrial apoptosis pathway in conditions of heart failure and hypertrophy [36,37]. A previous study by the present inventor indicated that the deletion of FoxO3a could inhibit maternal diabetes-induced apoptosis in cardiac progenitor cells in vivo [38]. The experimental data presented herein shows that FoxO3a gene deletion ameliorates maternal diabetes-induced CHDs by suppressing mitochondrial fragmentation and dysfunction. Thus, the inventor reveals the downstream effectors of FoxO3a, miR-140 and miR-195, in defective heart development.

Mitochondrial fusion, a prosurvival event, maintains mitochondrial homeostasis by removing dysfunctional mitochondria [39,40]. Cells lacking both Mfn1 and Mfn2 have completely fragmented mitochondria with no detectable mitochondrial fusion [40]. Mitochondrial fusion is important for the maintenance of mitochondrial morphology, cell growth, membrane potential, and respiration [39]. Reduced fusion could be a key factor contributing to diabetes- or miRNA-induced mitochondrial dysfunction. Maternal diabetes induces cellular dysfunction in cells required for cardiac septation leading to CHDs [17,24]. Enhanced mitochondrial fusion stimulates cell proliferation by promoting cell cycle progression [41]. Cells with double knockout of Mfn1 and Mfn2 proliferate much slower than their corresponding wild-type counterparts [42]. The experimental data presented herein shows that reduced Mfn1 and Mfn2 expression cause cellular dysfunction and alterations in cardiac septation leading to CHDs under conditions of maternal diabetes exposure and miRNA overexpression.

Teriflunomide (TERI) is a small molecule compound approved by U.S. FDA for the use in treatment of multiple sclerosis. However, studies also showed that teriflunomide could activate mitochondrial fusion. One study indicated that teriflunomide upregulates mitofusins and also induces mitochondrial elongation by depletion of the cellular pyrimidine pool secondary to the inhibition of dihydroorotate dehydrogenase [20]. Another study indicated that teriflunomide increases Mfn2 transcriptional activity and mitofusin mRNA levels in Hela cells [43]. Echinacoside is another small molecule compound currently being investigated for anti-apoptotic and neuroprotective effects [44,45]. This compound also can function as mitochondrial fusion activator. A study found that echinacoside selectively binds to the previously uncharacterized casein kinase 2 (CK2) α′ subunit (CK2α′) as a direct cellular target and allosterically regulates CK2α′ conformation to recruit basic transcription factor 3 (BTF3) to form a binary protein complex, and then the CK2α′/BTF3 complex facilitates β-catenin nuclear translocation to activate TCF/LEF transcription factors and stimulates transcription of the mitochondrial fusion gene Mfn2 [21]. These findings are consistent with the data presented herein. This data demonstrates that teriflunomide and echinacoside, acting as mitochondrial fusion activators, increase the expression levels of Mfn1 and Mfn2 and improve mitochondrial fusion in cardiomyocytes under diabetic conditions, in turn, prevent CHD formation in diabetic pregnancy.

Thus, and as detailed below, the present inventor revealed an epigenetic mechanism underlying maternal diabetes-induced CHDs. In summary, maternal diabetes-activated transcription factor FoxO3a increases miR-140 and miR-195, which in turn represses Mfn1 and Mfn2, leading to mitochondrial fusion defects and CHDs. Two mitochondrial fusion activators, teriflunomide and echinacoside, increase the expression level of Mfn1 and Mfn2, restore mitochondrial fusion, and prevent CHD formation. These two activators can thus be used in methods of preventing CHDs, methods of restoring or augmenting mitochondrial fusion, and methods of inducing or augmenting expression of Mfn1 and/or Mfn2 in diabetic pregnancy.

In a first embodiment, the invention is directed to methods of preventing congenital heart defects (CHDs) in an embryo of a pregnant diabetic female subject. These methods comprise administering to a pregnant diabetic female subject and/or an embryo of a pregnant diabetic female subject a therapeutically-effective amount of at least one mitochondrial fusion activator.

In a second embodiment, the invention is directed to methods of restoring or augmenting mitochondrial fusion in an embryo of a pregnant diabetic female subject. These methods comprise administering to a pregnant diabetic female subject and/or an embryo of a pregnant diabetic female subject a therapeutically-effective amount of at least one mitochondrial fusion activator. Mitochondrial fusion may be restored, for example, in cardiomyocytes.

In a third embodiment, the invention is directed to methods of inducing or augmenting expression of mitofusin 1 (Mfn1) and/or mitofusin 2 (Mfn2) in an embryo of a pregnant diabetic female subject. These methods comprise administering to a pregnant diabetic female subject and/or an embryo of a pregnant diabetic female subject a therapeutically-effective amount of at least one mitochondrial fusion activator.

In each of the relevant aspects and embodiments of the invention, the mitochondrial fusion activator may be one or more of teriflunomide (TERI), echinacoside (ECH), and other chemical or natural compounds which can activate mitochondrial fusion.

In each of the relevant aspects and embodiments of the invention, the congenital heart defect is one or more of cardiac septation defects including, but not limited to, Atrioventricular Septal Defect, Coarctation of the Aorta, Hypoplastic Left Heart Syndrome, Persistent truncus arteriosus, and Tetralogy of Fallot.

In each of the relevant aspects and embodiments of the invention, the methods may be practiced during the first and early second trimesters of humans, and during age E0.5 to E17.5 in mouse embryos.

The female subjects on which the methods of the invention may be practiced are female mammals, e.g. a human, a non-human primate, horse, cow, goat, sheep, a companion animal, such as a dog, cat or rodent, or other mammal.

In each of the relevant aspects and embodiments of the invention, the female subject that is diabetic has pre-gestational diabetes, gestational diabetes, type 1 diabetes or type 2 diabetes.

In each of the relevant aspects and embodiments of the invention, the “preventing”, “reducing” and “abrogating” is at least 50%, in comparison to a subject not undergoing the one of the methods of the invention. Each of the “preventing”, “reducing” and “abrogating” may also be at least 50%, at least 60%, at least 70%, at least 80%, at least 90%, at least 95%, at least 99%, or 100%, in comparison to a subject not undergoing the one of the methods of the invention.

In each of the relevant aspects and embodiments of the invention, the “restoring”, “augmenting” and “increasing” is at least 50%, in comparison to a subject not undergoing the one of the methods of the invention. Each of the “restoring”, “augmenting” and “increasing” may also be at least 50%, at least 60%, at least 70%, at least 80%, at least 90%, at least 95%, at least 99%, or 100%, in comparison to a subject not undergoing the one of the methods of the invention.

In each of the relevant aspects and embodiments of the invention, the “therapeutically-effective amount of at least one mitochondrial fusion activator” is an amount of mitochondrial fusion activator sufficient to achieve the goal of the method, i.e. preventing CHDs, restoring or augmenting mitochondrial fusion, or inducing or augmenting expression of Mfn1 and/or Mfn2. The therapeutically-effective amount of the mitochondrial fusion activator is a dose of between 0.1 mg/kg and 1000 mg/kg body weight of the subject.

The therapeutically-effective amount of TERI is a dose of between 0.1 mg/kg and 100 mg/kg, 0.5 mg/kg and 50 mg/kg, 1 mg/kg and 30 mg/kg or 5 mg/kg and 25 mg/kg body weight of the subject.

The therapeutically-effective amount of ECH is a dose of between 0.1 mg/kg and 100 mg/kg, 0.5 mg/kg and 50 mg/kg, 1 mg/kg and 30 mg/kg or 5 mg/kg and 25 mg/kg body weight of the subject.

In the methods of the present invention, the mitochondrial fusion activator may be administered to the subject or the embryo at a frequency that includes a single administration to achieve the methods of the invention. Alternatively, a suitable number of administrations includes 1, 2, 3, 4, 5, 6, 7, 8, 9 or 10 administrations of the mitochondrial fusion activator. When administered more than once, the frequency of administered may be every 15 minutes, every 30 minutes, every 45 minutes, every 60 minutes, every 75 minutes, every 90 minutes, every 105 minutes, or every 120 minutes, for example.

When administered to the subject, the mitochondrial fusion activator may be formulated, for example, for oral, sublingual, intranasal, intraocular, rectal, transdermal, mucosal, pulmonary, topical or parenteral administration. The mitochondrial fusion activator may be formulated in a suitable diluent, for example, phosphate buffered saline (PBS). Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection or infusion of drug formulations can be used to effect such administration.

The mitochondrial fusion activator may be administered to the embryo via any means suitable in the art of delivery to an embryo. The mitochondrial fusion activator may be formulated in a suitable diluent, for example, phosphate buffered saline (PBS). As a non-limiting example, intra-amniotic delivery (i.e. administration to the amniotic cavity of the embryo) via microinjections is a suitable means for administering mitochondrial fusion activators directly to an embryo and specifically targeting embryonic organs and cell types.

### III. Examples

### Materials and Methods

Animals. All animal procedures were approved by the Institutional Animal Care and Use Committee of the University of Maryland School of Medicine. The mouse diabetic embryopathy model has been described previously [11-13].

Wild-type (WT) (Stock No. #000664), miR-195^{f/f} (Stock No. #034659-JAX), and Tnnt2-Cre (Stock No. #024240) mice were purchased from the Jackson Laboratory (Bar Harbor, ME). FoxO3a KO mice on an FVB background was obtained from the Mutant Mouse Regional Resource Centers [46] and has been crossed back to C57BL/6J mice for 10 generations to generate FoxO3a KO mice on C57BL/6J background. FoxO3a^{+/-} males were mated with nondiabetic or diabetic FoxO3a^{+/-} females to generate WT, FoxO3a^{+/-}, and FoxO3a^{-/-} embryos. miR-195^{f/f} female mice were crossed with Tnnt2-Cre Transgenic (Tg) male mice to generate miR-195^{f/w}, Tnnt2-Cre male mice. The miR-195^{f/w}; Tnnt2-Cre males mated with nondiabetic or diabetic miR-195^{f/f} females to generate miR-195^{f/w}, miR-195^{f/f}, miR-195^{f/w}; Tnnt2-Cre, and miR-195^{f/f}, Tnnt2-Cre embryos. The miR-140 KO mice on a C57BL/6 background were a generous gift from Dr. Hiroshi Asahara in the Department of Molecular Medicine, at the Scripps Research Institute, La Jolla, CA [47]. miR-140^{+/-} male mice were mated with nondiabetic or diabetic miR-140^{+/-} female mice to generate WT, miR-140^{+/-}, and miR-140^{-/-} embryos.

The DNA construct for the Mfn1 transgenic (Mfn1-Tg) mice was driven by the cardiac muscle marker, Cardiac troponin T (Tnnt2), which is a gift from Dr. Terry Ann Van Dyke in the Department of Biological Sciences at University of Pittsburgh, PA. The DNA construct for the Mfn2 transgenic (Mfn2-Tg) mice was driven the nkx2.5 promoter. The DNA construct for the miR-195 and miR-140 double transgenic mice was driven by the nkx2.5 promoter (Nkx2.5-miR-195-miR-140-Tg), which was cloned based on a previous report [48]. All Tg animal lines were generated in the Genome Modification Facility at Harvard University using the C57BL/6J background as previously described [12]. The Mfn1-Tg or Mfn2-Tg male mice were mated with nondiabetic or diabetic WT female mice to generate WT and Mfn1-Tg or Mfn2-Tg embryos. The Nkx2.5-miR-195-miR-140-Tg male mice were mated with nondiabetic Mfn1-Tg female mice to generate WT, Nkx2.5-miR-195-miR-140-Tg, Mfn1-Tg, and Nkx2.5-miR-195-miR-140-Tg/Mfn1-Tg embryos.

Diabetic embryopathy model. The mouse diabetic embryopathy model has been described previously [49]. Briefly, female mice (WT, FoxO3a^{+/-}, miR-195^{f/f}, or miR-140^{+/-}) over ten weeks of age were intravenously (IV) injected with 75 mg/kg streptozotocin (STZ) for two consecutive days to induce diabetes. STZ was purchased from Sigma (St. Louis, MO) and dissolved in sterile 0.1 M citrate buffer (pH 4.5). Vehicle-injected nondiabetic female mice served as controls. Diabetes was defined as a 12-hour fasting blood glucose level of ≥250 mg/dl. There are no residual toxic effects caused by STZ in this animal model [13,23]. Male and female mice were paired at 3:00 P.M., and day 0.5 (E0.5) of pregnancy was established at noon of the day when a vaginal plug was present. E9.5 or E12.5 hearts were used for biochemical and molecular analyses because this is the critical time course of heart development. E17.5 hearts were used for morphological examination.

Mitochondrial fusion activator treatment. Teriflunomide was purchased from Millipore Sigma (Cat #SML0936) and dissolved in a solution made of 10% dimethyl sulfoxide (DMSO)+90% corn oil. Echinacoside was purchased from Millipore Sigma (Cat #07668) and dissolved in water. Female mice were injected intraperitoneally with teriflunomide or echinacoside from E7.5-E12.5 at a dose of 15 mg/kg. Vehicle injections served as controls. At E17.5, mice were killed and major vessels of embryonic hearts were examined. The hearts were fixed in 4% paraformaldehyde (PFA) for sectioning to examine cardiac chamber defects. At E13.5, embryonic hearts were collected for teriflunomide or echinacoside measurement by high-performance liquid chromatography-mass spectrometry (HPLC-MS) in the Mass Spectrometry Facility of the University of Maryland at College Park.

India ink injection and hematoxylin-eosin (H & E) staining. Diluted India ink (1:100) was injected into the left ventricle and perfused through the vascular system. E17.5 embryonic hearts were collected and fixed in 4% paraformaldehyde (PFA) overnight at 4° C., and then rinsed with PBS 3 times, followed by a series of graded ethanol baths to dehydrate the hearts. Xylene clearance was performed before the hearts were embedded in paraffin. All the paraffin blocks were cut into 5-μm sections. After deparaffinization and rehydration, all specimens underwent Hematoxylin-eosin (H & E) staining in a standard procedure. All heart sections were photographed and examined for heart defects.

Cell culture and transfection. The cardiac myoblast cell line H9C2 was obtained from Sigma-Aldrich. The primary cardiomyocytes were isolated from the E12.5 mouse embryonic hearts using the Pierce Primary Cardiomyocyte Isolation Kit (Thermo Fisher Scientific, Rockford, IL). The cells were cultured in DMEM medium supplemented with 10% fetal bovine serum, 100 U/ml penicillin, and 100 μg/ml streptomycin at 37° C. in a humidified atmosphere of 5% CO₂. Cells were either cultured under normal glucose (5 mM) or high glucose (25 mM) conditions which were consistent with the fast blood glucose level in nondiabetic (90 mg/dL) or diabetic (450 mg/dL) mice. Cells were transfected with plasmids carrying mitochondrial matrix-target photoactivable green fluorescent protein (ad-Mt-PAGFP), FoxO3a constitutive active (FoxO3a^{-/-} CA) or FoxO3a dominant negative (FoxO3a^{-/-} DN), a miR-195 mimic, a miR-195 inhibitor, a miR-140 mimic, or a miR-140 inhibitor (Life Technologies, Gaithersburg, MD).

Mitochondrial length measurement. E12.5 cardiomyocytes were isolated and plated in a chamber slide. After a culture of 24 hours, cells were transfected with a plasmid carrying mitochondrial matrix-targeted photoactive GFP (mito-PAGFP) for 48 hours and then fixed with 4% PFA, followed by staining with a myomesin antibody (1:200, Santa Cruz Biotechnology, Dallas, TX) staining. Cells were imaged using a laser scanning confocal microscope (Zeiss LSM 510 META). Image J software was used to measure the mitochondrial length. The percentage of mitochondria with a length between 1 to 2 μm or >2 μm was analyzed for every one hundred mitochondria in a representative area.

Mitochondrial fusion assay. A mito-PAGFP-based mitochondrial fusion assay was performed using a Zeiss LSM 510 META confocal microscope (Zeiss MicroImaging) as previously described [19,50,51]. Briefly, after the acquisition of a preactivation image of cells, a circular region of interest (˜5 μm in diameter) was photoactivated by brief irradiation with 351/364-nm light (Coherent Enterprise Ion Laser 80.0 mW), which was followed by time-lapse imaging with a 488-nm excitation light (488-nm Argon Ion Laser 25.0 mW). Fifty postactivation images were collected with the interval between images set to ˜30 seconds. Images were acquired and analyzed with the ZEN 2009 image acquisition software (Zeiss MicroImaging). For quantification of the dynamics of mitochondrial fusion, the time-lapse images of cells were analyzed using the NIH ImageJ software. Briefly, all the time-lapse images were converted to binary images before the measurement of pixel intensity. The pixels in the first postactivation images in each group were assigned a value of 1, and the pixel intensity of other time-dependent postactivation images was normalized by dividing the pixel intensity by those of the first ones. The results from multiple cells were aligned and the time-dependent results were averaged together.

Immunofluorescent staining. E9.5 whole embryo sections and E12.5 embryonic heart sections were used for immunofluorescent staining. The tissue processing and embedding were the same as described in the “India ink injection and tissue processing” section. For cell staining, the primary cardiomyocytes were fixed in 4% PFA for 15 min prior to blocking. Deparaffinized tissue sections or cells were blocked for 2 hours in PBS with 10% donkey serum and incubated with antibodies against phosphor-Histone H3 (1:200, Cell Signaling Technology, Danvers, MA), Myomesin, and FoxO3a (1:1000, Invitrogen, Carlsbad, CA) overnight at 4° C. After three times of wash with PBS, samples were incubated with Alexa-Fluor 488 or Alexa-Fluor 594 secondary antibodies (1:1000, Invitrogen, Carlsbad, CA) for 2 hours at room temperature, followed by DAPI (1:1000, Invitrogen, Carlsbad, CA) counterstaining, and then mounted with aqueous mounting medium (Sigma, St Louis, MO). Confocal immunofluorescent images were recorded by a laser scanning microscope (LSM 510 META; Zeiss, Germany).

Immunoblotting. Immunoblotting was performed as previously described [52,53]. Protein was extracted from E12.5 embryonic hearts by sonication in ice-cold lysis buffer (Cell Signaling Technology, Beverly, MA) with a protease inhibitor cocktail (Sigma-Aldrich). Equal amounts of protein from each group were resolved by SDS-polyacrylamide gel electrophoresis, transferred onto PVDF membrane, and immunoblotted with primary antibodies at 1:1000 dilutions in 5% nonfat milk. The detailed antibody information is provided in online supplementary materials. Following the primary antibody incubation, the membranes were exposed to goat anti-rabbit or anti-mouse secondary antibodies. The signals were detected by the SuperSignal West Femto Maximum Sensitivity Substrate kits (Thermo Scientific, Rockford, IL). The intensities of target protein bands were assessed by densitometry and normalized to those of β-actin (Abcam, Cambridge, MA). All experiments were repeated three times with the use of independently prepared tissue lysates. All uncropped immunoblotting images are present in online supplementary materials.

Real-time quantitative PCR (RT-qPCR). Total RNA was isolated from E12.5 embryonic hearts and cells using TRIzol reagent (Ambion) and reverse transcribed to cDNA using the QuantiTect Reverse Transcription Kit (Qiagen). Reverse transcription for miRNA was performed using the qScript microRNA cDNA Synthesis Kit (Quanta Biosciences). RT-qPCR of Mfn1, Mfn2, FoxO3a, and β-actin were performed using the Maxima SYBR Green/ROX qPCR Master Mix assay (Thermo Fisher Scientific, Waltham, MA). RT-qPCR of pri-miR-195, miR-195, pri-miR-140, miR-140, and U6 were performed using the TB Green® Advantage® qPCR Premix (Takara). RT-qPCR and subsequent calculations were performed by a StepOnePlus Real-Time PCR System (Applied Biosystem). Primer sequences are listed in online supplementary materials.

Biotin-labeled pulldown assay. The pulldown assay was conducted following the previously described methods [54]. The biotin-labeled miR-195, miR-140, or negative control (Dharmacon Lafayette, CO) was transfected into H9C2 cells for 48 hours, and whole-cell lysates were collected. Cell lysates were mixed with streptavidin coupled Dynabeads (Invitrogen) and incubated at 4° C. on a rotator overnight. After the beads were washed thoroughly, the bead-bound RNA was isolated and reverse transcribed followed by RT-qPCR analysis. Input RNA was extracted and served as a control.

RNA immunoprecipitation. RNA immunoprecipitation was performed using the EZ-Magna RIP™ RNA-Binding Protein Immunoprecipitation Kit (EMD Millipore). Briefly, whole E12.5 embryonic hearts were harvested and lysed. The lysates were mixed with magnetic beads and Argonaute 2 (AGO2) antibody (Abcam) and incubated at 4° C. on a rotator overnight. After the beads were washed thoroughly, the bead-bound RNA was isolated and subjected to reverse transcription followed by RT-qPCR analysis. Input RNA was extracted and served as a control.

Luciferase activity measurement. The binding sites of the transcription factor FoxO3a, which potentially regulates miR-195 and miR-140 promoter activity, were predicted using online prediction tools (http://jaspar.genereg.net). One FoxO3a binding site was identified in the miR-195 promoter region and three FoxO3a binding sites in the miR-140 promoter region. The miR-195 or the miR-140 promoter was subcloned and inserted into a pGL4.10 Luciferase Reporter Vector (Promega). Co-transfection of FoxO3a constitutively active (CA-FoxO3a) or dominant-negative (DN-FoxO3a) vector with the pGL4.1-miR195 promoter vector or pGL4.1-miR140 promoter vector into H9C2 cells was performed using Lipofectamine 2000 (Invitrogen), and the cells were cultured for 48 hours with the treatment of normal glucose (5 mM) or high glucose (25 mM). The CA-FoxO3a vector (Addgene plasmid #1788) and DN-FoxO3a vector (Addgene plasmid #1797) were gifts from M. Greenberg, in the Department of Neurobiology, at Harvard Medical School [55]. Cells were harvested and lysed using lysis buffer from the Dual-Luciferase Assay System (Promega). Renilla luciferase activities were used as the internal control.

TUNEL assay. The TUNEL assay was performed using the ApopTag Fluorescein In Situ Cell Death Detection Kit (S7165, Millipore, Billerica, MA) as described previously [23]. E9.5 whole embryo sections or E12.5 embryonic heart sections were subject to the TUNEL procedure according to the manufacturer's protocol. Three embryos from three different dams (n=3) in each group were used, and two sections per embryo were examined. The percentage of apoptotic cells was calculated as the number of apoptotic cells divided by the total number of cells in a selected area.

Flow cytometry assay. The mitochondrial membrane potential was analyzed using the JC-1 dye (Invitrogen). Briefly, cells were harvested and then incubated with JC-1 dye (1 μg/ml in medium) at 37° C. for 15 min. After three times of wash with PBS, the stained cells were analyzed by a flow cytometer. The ratio of fluorescence intensity (red to green) represented the mitochondrial membrane potential.

Statistical analysis. All experiments were repeated in triplicate. Specifically, for immunostaining, three embryonic samples from three litters were stained for each group and average signal intensity was calculated. For immunoblotting, one embryonic heart from one litter in each group was used for one run. Each experiment was repeated three times with three embryonic hearts from three different litters in each group. For RT-qPCR, three embryonic hearts from different litters in each group were analyzed. Data are presented as the mean±standard derivation (SD). Student's t test was used for two group comparison. One-way variance (ANOVA) with Tukey's post-hoc test for multiple pairwise comparisons was used for comparisons of more than two groups. CHD incidence is presented as count with percentage and compared using chi-square test. Differences were considered statistically significant when P<0.05.

### Results

Pharmacological activation of mitochondrial fusion ameliorates CHDs. Heart cells, especially cardiomyocytes, are enriched in mitochondria [14]. Mitochondrial fusion and fission dynamics play an important role in the regulation of cardiomyocyte viability [15]. Because abrogating mitochondrial fusion leads to CHDs [16] similar to those observed in diabetic pregnancy [17], mitochondrial morphology and fusion were examined in cardiomyocytes of embryonic mouse hearts from normal healthy dams and those with maternal diabetes induced before mating by treatment with streptozotocin (STZ).

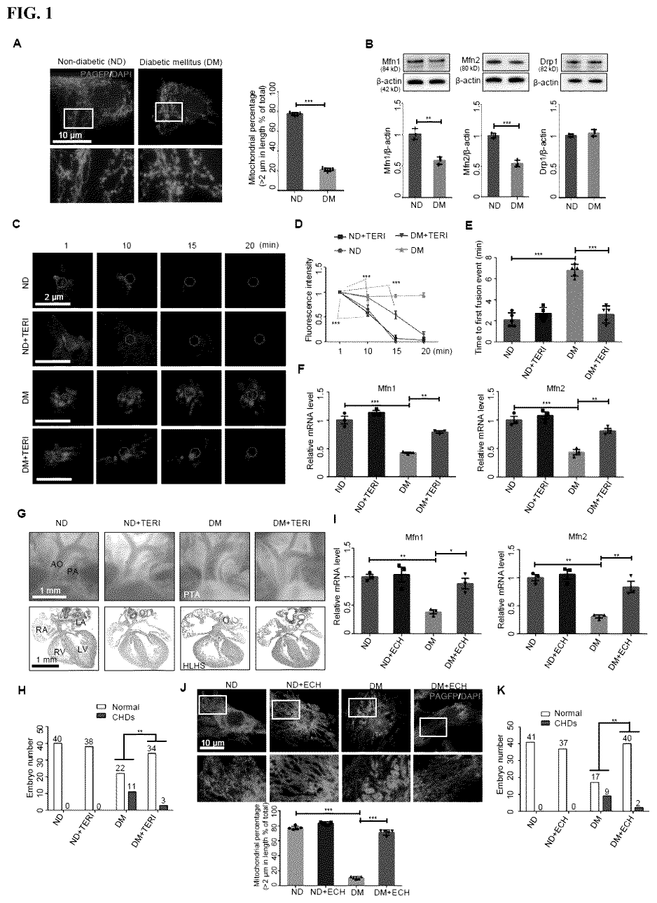

Under non-diabetic conditions, mitochondria in embryonic cardiomyocytes exhibited tubular morphology whereas maternal diabetes triggered fragmented and small spherical mitochondria (FIG. 1A). The sizes of mitochondria in cardiomyocytes from embryos exposed to maternal diabetes were significantly smaller than those from non-diabetic dams (FIG. 1A). Mitochondrial fusion and fission modulate mitochondrial morphology dynamics [18]. The proteins Mfn1 and Mfn2 are essential for mitochondrial fusion, and dynamin-related protein 1 (Drp1) induces mitochondrial fission [18]. Maternal diabetes was associated with lower Mfn1 and Mfn2 expression in the developing heart compared to non-diabetic controls but did not affect Drp1 expression (FIG. 1B). These findings suggest that maternal diabetes represses mitochondrial fusion leading to reduced mitochondrial sizes. Because the double ablation of Mfn1 and Mfn2 in the developing heart results in complex CHDs and lethality at middle gestation [16], it was hypothesized that reduced levels of Mfn1 and Mfn2, and the consequent repression of mitochondrial fusion are responsible for maternal diabetes-induced CHDs.

Mitochondrial fusion was assessed in primary cardiomyocytes from embryonic day 12.5 (E12.5) hearts using a validated assay that monitors the redistribution of mitochondrial matrix-target photoactivable green fluorescent protein (mito-PAGFP) [19]. Time-lapse imaging showed the mito-PAGFP intensity in cardiomyocytes from non-diabetic dams began to be significantly reduced at an average of 10 min and diminished at 15 min after GFP photoactivation (FIG. 1C, 1D), indicating active mitochondrial fusion because of the diffusion of mito-PAGFP into non-GFP mitochondria [19]. The first mitochondrial fusion event, defined at the time of mito-PAGFP flow into a non-GFP mitochondrion (FIG. 1E), occurred at 2 min post-photoactivation in the non-diabetic group. In contrast, the mito-PAGFP intensity in cardiomyocytes from diabetic dams remained unchanged during the entire 20 min imaging period and mitochondrial fusion did not occur in these cells (FIG. 1C, 1D).

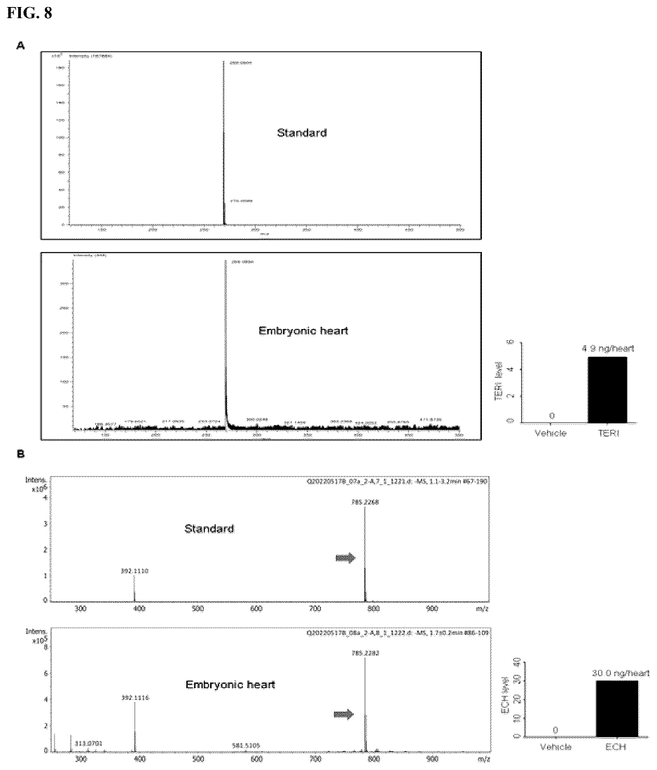

Treatment with the mitochondrial fusion activator teriflunomide [20], an FDA-approved drug for treating relapsing multiple sclerosis, restored Mfn1 and Mfn2 expression and mitochondrial fusion suppressed by maternal diabetes (FIG. 1C, 1D, 1E, 1F; FIG. 8A). Teriflunomide did not further accelerate mitochondrial fusion under non-diabetic conditions, and mitochondrial fusion appeared to reach the maximum level (FIG. 1C, 1D). Consequently, teriflunomide significantly ameliorated CHD formation under diabetic conditions (FIG. 1G, 1H; Table 1).

TABLE 1
Teriflunomide ameliorates CHDs in diabetic pregnancy

| | | Glucose level | Total | |
|---|---|---|---|---|
| Experimental group | | (mg/dl) | embryos | CHDs |
| ND | Vehicle (n = 5) | 103.2 ± 17.1 | 40 | 0 |
| | TERI (n = 5) | 116.6 ± 7.4 | 38 | 0 |
| DM | Vehicle (n = 5) | 410.8 ± 22.3 | 33 | 11 (33.3%)* |
| | TERI (n = 5) | 397.0 ± 24.0 | 37 | 3 (8.1%) |

ND: nondiabetic;
DM: diabetic mellitus;
CHDs: congenital heart defects;
TERI: teriflunomide;
Vehicle: 10% DMSO + 90% corn oil;
*indicates significant differences (P < 0.05) compared with other groups by chi-square test.

Another mitochondrial fusion activator echinacoside [21], a naturally occurring small molecule compound, also restored Mfn1 and Mfn2 expression (FIG. 1I, 8B) and mitochondrial sizes (FIG. 1J), and alleviated maternal diabetes-induced CHDs (FIG. 1K; Table 2).

TABLE 2
Echinacoside mitigates CHD formation in diabetic pregnancy

| | | Glucose level | Total | |
|---|---|---|---|---|
| Experimental group | | (mg/dl) | embryos | CHDs |
| ND | Vehicle (n = 5) | 111.5 ± 13.2 | 41 | 0 |
| | ECH (n = 5) | 109.2 ± 9.6 | 37 | 0 |
| DM | Vehicle (n = 4) | 408.5 ± 32.0 | 26 | 9 (34.6%)* |
| | ECH (n = 5) | 418.0 ± 27.2 | 42 | 2 (4.8%) |

ND: nondiabetic;
DM: diabetic mellitus;
CHDs: congenital heart defects;
ECH: echinacoside;
Vehicle: distilled water;
*indicates significant differences (P < 0.05) compared with other groups by chi-square test.

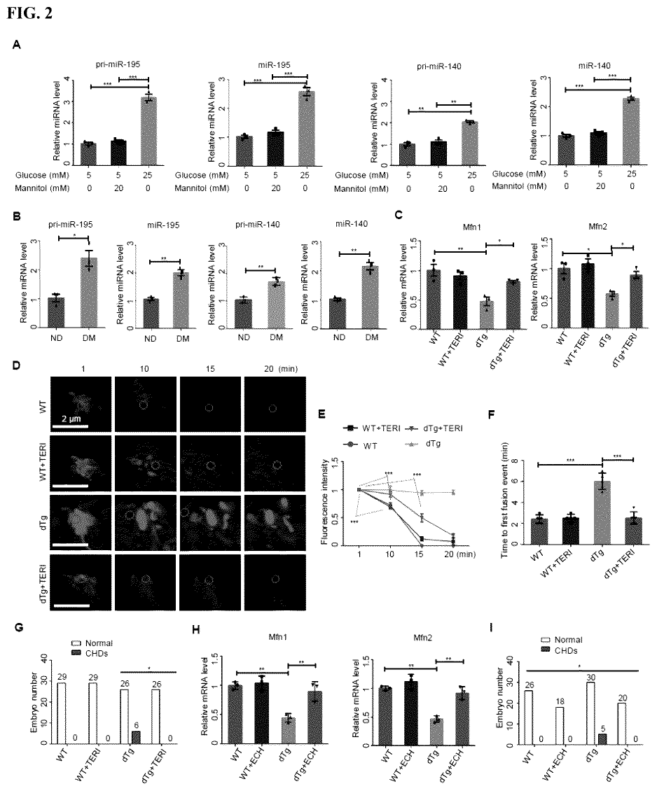

Next, molecular intermediates that mediate the teratogenicity of maternal diabetes in the developing heart were sought. High glucose conditions in vitro and maternal diabetes in vivo significantly increased the expression of miR-195 and miR-140 (FIG. 2A, 2B). Several miRNAs negatively regulate mitochondrial fusion [22]. Transgenic overexpression of both miR-195 and miR-140 in the developing heart mimicked the effects of maternal diabetes on the repression of Mfn1 and Mfn2, the blockage of mitochondrial fusion, and the induction of CHDs (FIG. 2C, 2D, 2E, 2F, 2G). Teriflunomide blocked miR-195/140-suppressed mitochondrial fusion and thus reduced CHD formation (FIG. 2D, 2E, 2F, 2G; Table 3). Likewise, echinacoside sustained Mfn1 and Mfn2 expression and ameliorated CHDs induced by miR-195/140 upregulation (FIG. 2H, 2I; Table 4). Thus, impaired mitochondrial fusion leads to CHD formation and fusion activators can reduce the incidence of CHDs induced by maternal diabetes and upregulation of specific miRNAs.

TABLE 3
Teriflunomide blocks CHD formation induced by transgenic
overexpression of miR-195 and miR-140

| | | | Total | |
|---|---|---|---|---|
| Experimental group | | Genotype | embryos | CHDs |
| Control | Nkx2.5-miR-195- | WT | 29 | 0 |
| | miR140-Tg ♂ × | Tg | 32 | 6 (18.6%)* |
| | WT♀(n = 8) | | | |
| TERI | Nkx2.5-miR-195- | WT | 29 | 0 |
| | miR140-Tg ♂ × | Tg | 26 | 0 |
| | WT♀(n = 7) | | | |

TERI: teriflunomide;
Mice in the control group were i.p. injected with vehicle (10% DMSO + 90% corn oil).
WT: wild type;
Tg: transgenic;
CHDs: congenital heart defects;
*indicates significant differences (P < 0.05) compared with other groups by chi-square test.

TABLE 4
Echinacoside blocks CHD formation induced by transgenic
overexpression of miR-195 and miR-140

| | | | Total | |
|---|---|---|---|---|
| Experimental group | | Genotype | embryos | CHDs |
| Control | Nkx2.5-miR-195- | WT | 26 | 0 |
| | miR140-Tg♂ × | Tg | 35 | 5 (14.3%)* |
| | WT♀(n = 10) | | | |
| ECH | Nkx2.5-miR-195- | WT | 18 | 0 |
| | miR140-Tg♂ × | Tg | 20 | 0 |
| | WT♀(n = 5) | | | |

ECH: echinacoside;
Mice in the control group were i.p. injected with vehicle (10% DMSO + 90% corn oil).
WT: wild type;
Tg: transgenic;
CHDs: congenital heart defects;
*indicates significant differences (P < 0.05) compared with other groups by chi-square test.

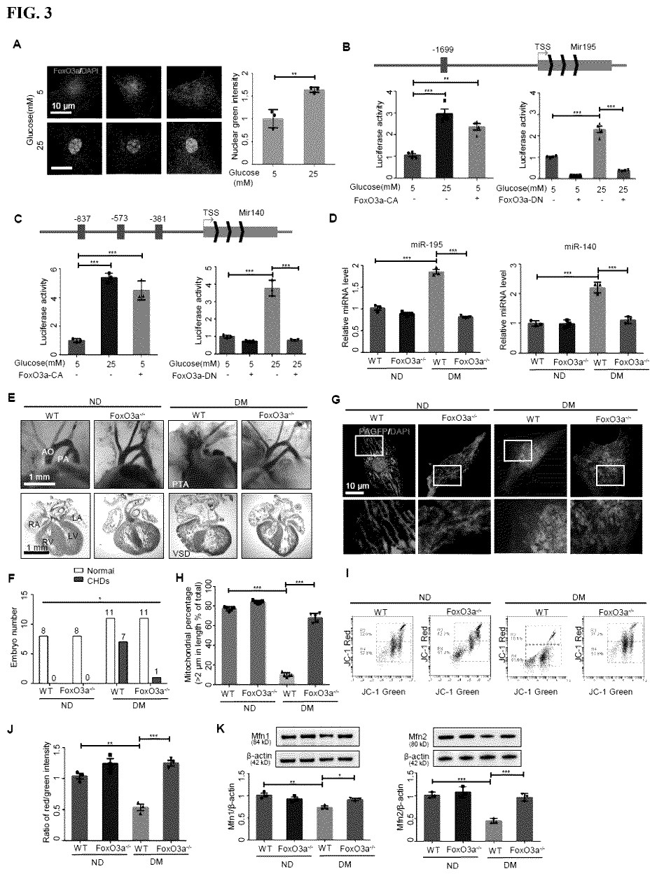

FoxO3a upregulates miR-195/140 and represses mitochondrial fusion. To determine how maternal diabetes induces teratogenic miR195/miR-140 expression in the heart, the transcription factor FoxO3a studied. FoxO3a is critically involved in diabetes-induced embryonic noncardiac anomalies [23]. High-glucose conditions activate FoxO3a by triggering its nuclear translocation in embryonic cardiomyocytes (FIG. 3A). Constitutively active (CA)-FoxO3a mimicked the high glucose-increased Mir195 and Mir140 promoter activity, and the dominant negative (DN)-FoxO3a inhibited the promoter activity (FIG. 3B, 3C). FoxO3a germline deletion in mice abolished the maternal diabetes-increased expression of miR-195 and miR-140 (FIG. 3D), reduced CHD formation (FIG. 3E, 3F; Table 5), restored mitochondrial lengths (FIG. 3G, 3H) and function (FIG. 31, 3J), and reversed the downregulation of Mfn1 and Mfn2 (FIG. 3K). These findings indicated that hyperglycemia-induced activation of the transcriptional factor FoxO3a leads to the upregulation of specific miRNAs and the development of CHDs in diabetic pregnancy by repressing mitofusin gene expression.

TABLE 5
Deletion of Foxo3a ameliorates maternal diabetes-induced CHDs

| | | Glucose | | Total | |
|---|---|---|---|---|---|
| Experimental group | | level(mg/dl) | Genotype | embryos | CHDs |
| ND | FoxO3a^{+/-}♂ × | 137.5 ± 7.1 | WT | 8 | 0 |
| | FoxO3a^{+/-}♀ (n = 7) | | FoxO3a^{+/-} | 14 | 0 |
| | | | FoxO3a^{-/-} | 8 | 0 |
| DM | FoxO3a^{+/-}♂ × | 440.2 ± 15.6 | WT | 18 | 7^{a} (38.9%)* |
| | FoxO3a^{+/-}♀ (n = 12) | | FoxO3a^{+/-} | 28 | 7^{b} (25.0%) |
| | | | FoxO3a^{-/-} | 12 | 1^{c} (8.3%) |

ND: nondiabetic;
DM: diabetic mellitus;
WT: wild-type;
+/−: heterozygous;
−/−: homozygous;
CHDs: congenital heart defects;
^{a}includes 4 ventricular septum defect (VSD), 2 hypoplastic left heart syndrome (HLHS), and 1 persistent truncus arteriosus (PTA);
^{b}includes 6 VSD and 1 transposition of the great arteries (TGA);
^{c}includes 1 VSD;
*indicates significant differences (P < 0.05) compared with the ND groups and the DM FoxO3a^{-/-} by chi-square test.

miR-195 deletion restores mitochondrial fusion by de-repressing Mfn2. The gain-of-function of miR-195 and miR-140 in transgenic (Tg) mice resembles diabetes-induced CHDs (FIG. 2G, 2I). The next goal was to determine the individual roles of miR-195 and miR-140 in the induction of CHDs in diabetic pregnancy using loss-of-function approaches.

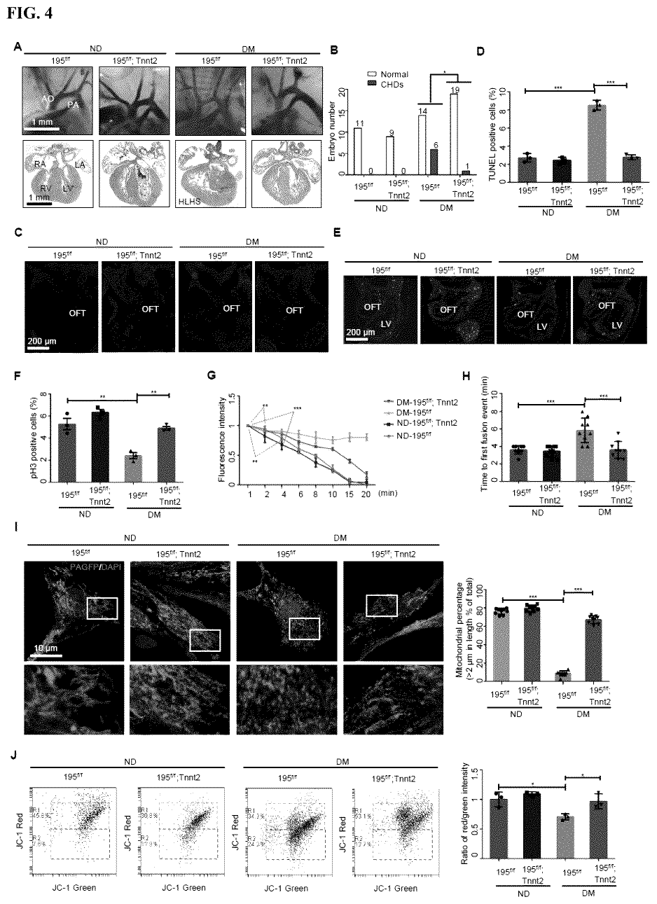

Cardiomyocyte-specific Mir195 deletion alleviated diabetes-induced CHDs (FIG. 4A, 4B; Table 6), cardiac cell apoptosis (FIG. 4C, 4D) and restored cell proliferation in the embryonic hearts (FIG. 4E, 4F). Because double Tg expression of miR-195 and miR-140 inhibited mitochondrial fusion (FIG. 2D, 2E, 2F), it is plausible that miR-195 mediates the inhibitory effect of maternal diabetes on mitochondrial fusion. Indeed, Mir195 deletion prevented maternal diabetes-inhibited mitochondrial fusion, which was manifested by the fast diffusion and eventual disappearance of mito-PAGFP and the restoration of the first mitochondrial fusion event in cardiomyocytes (FIG. 4G, 4H). Consequently, miR-195 deficiency restored mitochondrial lengths and function in cardiomyocytes (FIG. 41, 4J).

TABLE 6
Mir195 deletion in the developing heart reduces maternal diabetes-induced CHDs

| | | Glucose | | Total | |
|---|---|---|---|---|---|
| Experimental Group | | level (mg/dl) | Genotype | embryos | CHDs |
| ND | miR-195^{f/w}; Tnnt2 ♂ × | 118.2 ± 17.3 | miR-195^{f/w} | 9 | 0 |
| | miR-195^{f/f} ♀ (n = 6) | | miR-195^{f/f} | 11 | 0 |
| | | | miR-195^{f/w}; Tnnt2 | 10 | 0 |
| | | | miR-195^{f/f}; Tnnt2 | 9 | 0 |
| DM | miR-195^{f/w}; Tnnt2 ♂ × | 424.6 ± 27.8 | miR-195^{f/w} | 21 | 5^{a} (23.9%) |
| | miR-195^{f/f} ♀ (n = 11) | | miR-195^{f/f} | 20 | 6^{b} (30%)* |
| | | | miR-195^{f/w}; Tnnt2 | 20 | 5^{c} (25%) |
| | | | miR-195^{f/f}; Tnnt2 | 20 | 1^{a} (5%) |

ND: nondiabetic;
DM: diabetic mellitus;
Tnnt2: Tnnt2-Cre;
f/w: heterozygous floxed;
f/f: homozygous floxed;
CHDs: congenital heart defects;
^{a}includes 5 ventricular septum defect (VSD), with one of them combining persistent truncus arteriosus (PTA);
^{b}includes 5 VSD and 1 hypoplastic left heart syndrome (HLHS);
^{c}includes 4 VSD and 1 HLHS;
^{d}includes 1 VSD;
*indicates significant differences (P < 0.05) compared with the ND groups and the DM miR-195^{f/f}; Tnnt2 group by chi-square test.

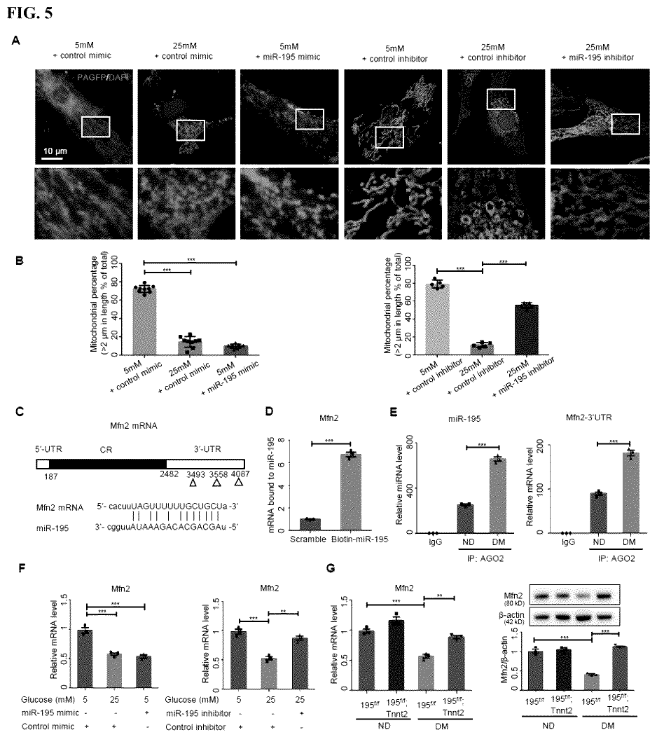

It was further shown that miR-195 could directly reduce mitochondrial lengths by decreasing Mfn2 expression. While a miR-195 mimic replicated the high glucose-reduced mitochondrial lengths in cardiomyocytes, a miR-195 inhibitor blocked the shortening of mitochondrial lengths in high-glucose conditions (FIG. 5A, 5B). A pull-down assay using biotin-labeled miR-195 demonstrated the direct binding of miR-195 to Mfn2 mRNA (FIG. 5C, 5D), and miR-195 and Mfn2 mRNA were coenriched in the AGO2 RNA-induced silencing complex in embryonic hearts exposed to maternal diabetes (FIG. 5E). However, miR-195 binding sites were not identified in Mfn1 mRNA. The miR-195 mimic resembled high-glucose conditions in repressing Mfn2 mRNA expression, and the miR-195 inhibitor blocked the high glucose-induced inhibition of Mfn2 expression (FIG. 5F). Deletion of miR-195 in the heart reversed the decrease in Mfn2 protein and mRNA levels in embryos exposed to maternal diabetes (FIG. 5G). These findings establish that the inhibition of Mfn2 by miR-195 and miR-195 deficiency in cardiomyocytes is sufficient to block the teratogenicity of diabetes and the mitochondrial fusion inhibition elicited by maternal diabetes.

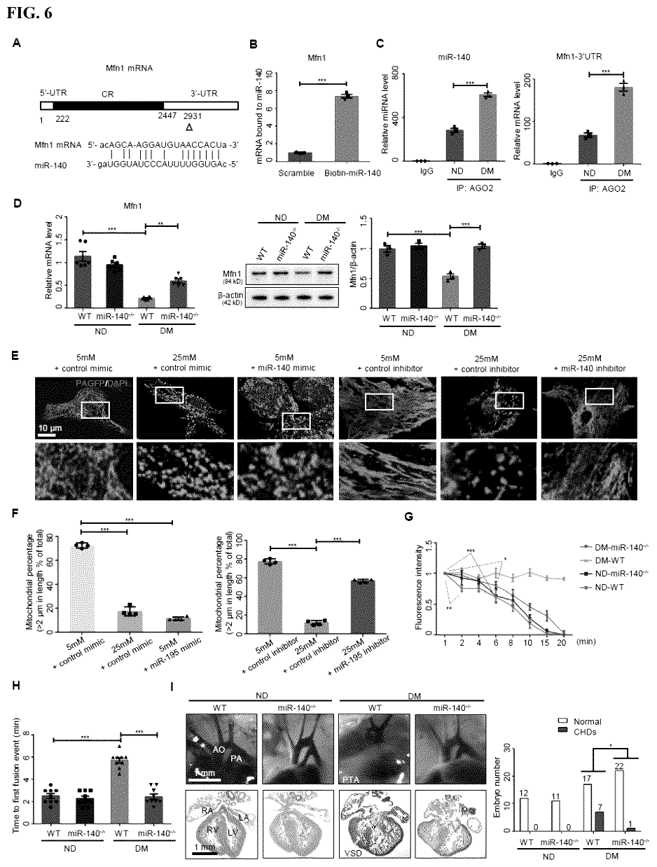

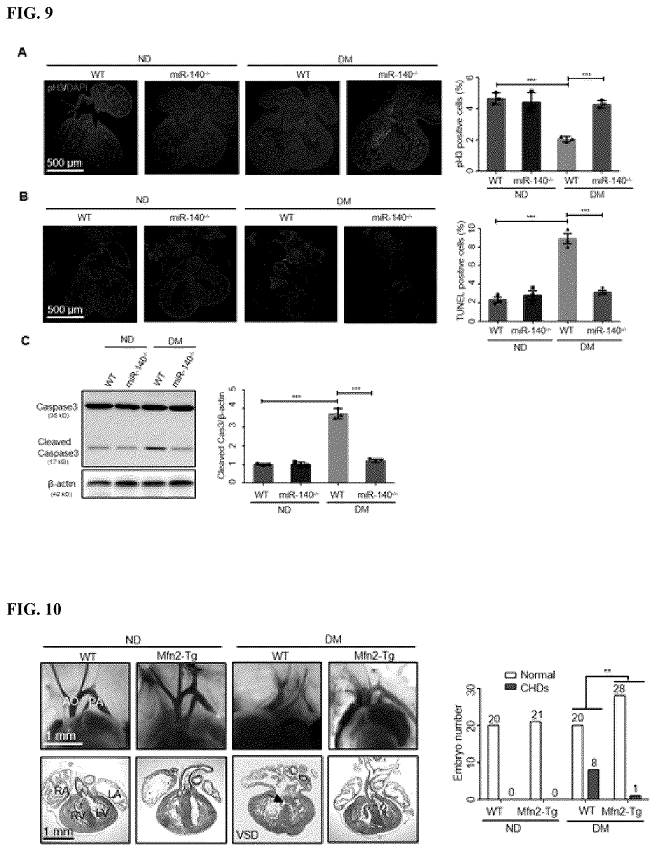

miR-140 deficiency removes the blockage of Mfn1 and mitochondrial fusion. miR-140 binds to Mfn1 mRNA in its 3′ untranslated region (3′-UTR) (FIG. 6A, 6B). miR-140 and Mfn1 mRNA were coenriched in the mRNA degradation AGO2 complex under maternal diabetic conditions (FIG. 6C), suggesting that Mfn1 is a miR-140 target. miR-140 deletion blocked the downregulation of Mfn1 at both the mRNA and protein levels in embryonic hearts exposed to diabetes (FIG. 6D). Because miR-140 degrades Mfn1, it should inhibit mitochondrial fusion, leading to mitochondrial fragmentation. Indeed, the effect of a miR-140 mimic resembled high-glucose conditions, as the mitochondrial lengths were reduced. Additionally, a miR-140 inhibitor ablated mitochondrial length shortening in embryonic cardiomyocytes under high-glucose conditions (FIG. 6E, 6F). The inhibition of mitochondrial fusion was abrogated by miR-140 deletion, which was manifested by faster dispersal of mito-PAGFP and a shorter time to reach the first mitochondrial fusion event in cardiomyocytes from diabetic dams compared with non-diabetic dams (FIG. 6G, 6H). Consequently, miR-140 deletion reduced the incidence of CHDs in diabetic pregnancy (FIG. 61; Table 7), and recovered cell proliferation and survival in the developing heart (FIG. 9A, 9B, 9C). Thus, miR-140 mediates the teratogenic effect of maternal diabetes by suppressing mitochondrial fusion leading to formation of CHDs.

TABLE 7
Mir140 deletion reduces maternal diabetes-induced CHDs

| | | Glucose | | Total | |
|---|---|---|---|---|---|
| Experimental group | | level (mg/dl) | Genotype | embryos | CHDs |
| ND | miR-140^{+/-}♂ × | 121.0 ± 12.7 | WT | 12 | 0 |
| | miR-140^{+/-} ♀ | | miR140^{+/-} | 21 | 0 |
| | (n = 6) | | miR140^{-/-} | 11 | 0 |
| DM | miR-140^{+/-}♂ × | 431.6 ± 22.6 | WT | 24 | 7^{a} (29.1%)* |
| | miR-140^{+/-} ♀ | | miR140^{+/-} | 31 | 6^{b} (19.4%) |
| | (n = 12) | | miR140^{-/-} | 23 | 1^{c} (4.4%) |

ND: nondiabetic;
DM: diabetic mellitus;
WT: wild-type;
+/−: heterozygous;
−/−: homozygous;
CHDs: congenital heart defects;
^{a}includes 6 ventricular septum defect (VSD), with one of them combining persistent truncus arteriosus (PTA), and 1 transposition of the great arteries (TGA);
^{b}includes 5 VSD and 1 TGA;
^{c}includes 1 VSD;
*indicates significant differences (P < 0.05) compared with the ND groups and the DM miR140^{-/-} group by chi-square test.

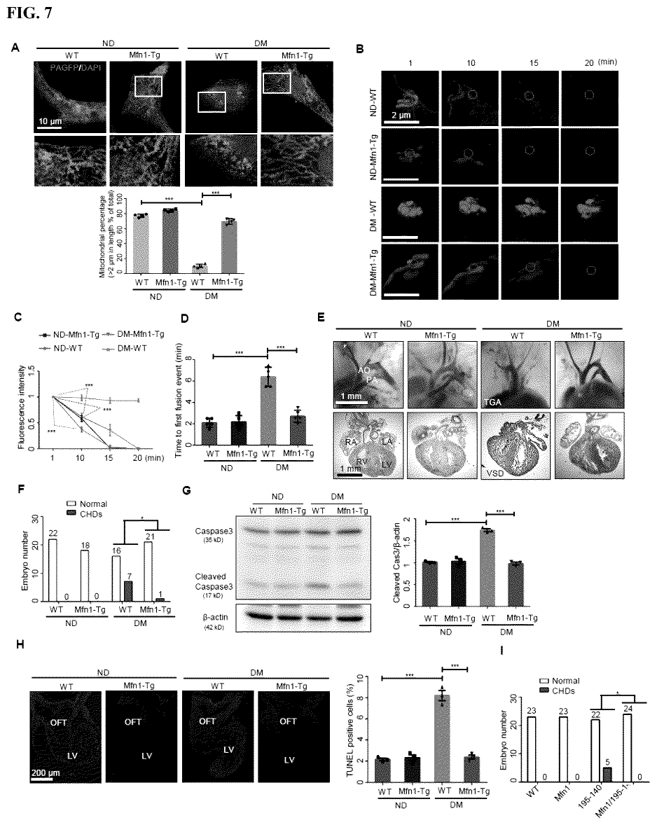

Restoring Mfn1 or Mfn2 expression abrogates the teratogenicity of maternal diabetes. The Tg overexpression of Mfn1 in the developing heart prevented the shortening of mitochondrial lengths (FIG. 7A) and reversed the inhibition of the dispersal of mito-PAGFP and the first mitochondrial fusion event in cardiomyocytes (FIG. 7B, 7C, 7D). There was a significant reduction in CHDs in embryos exposed to diabetes under the condition of Mfn1 Tg overexpression (FIG. 7E, 7F; Table 8). Furthermore, Mfn1 overexpression blunted maternal diabetes-induced caspase 3 cleavage and cell apoptosis (FIG. 7G, 7H). This was particularly true in the outflow tract, which is critical for cardiac septation. Mfn1 overexpression also blocked the formation of CHDs under the conditions of miR-140 and miR-195 overexpression (FIG. 71; Table 9), suggesting that maintaining the proper level of Mfn1 is essential for normal cardiac morphogenesis.

TABLE 8
Restoring Mfn1 expression in the developing heart
abrogates the teratogenicity of maternal diabetes

| | | Glucose level | | Total | |
|---|---|---|---|---|---|
| Experimental group | | (mg/dl) | Genotype | embryos | CHDs |
| ND | Mfn1-Tg ♂ × WT ♀ | 127.4 ± 9.2 | WT | 22 | 0 |
| | (n = 5) | | Tg | 18 | 0 |
| DM | Mfn1-Tg ♂ × WT ♀ | 433 ± 15.5 | WT | 23 | 7^{a} (30.4%)* |
| | (n = 8) | | Tg | 22 | 1^{b} (4.6%) |

ND: nondiabetic;
DM: diabetic mellitus;
WT: wild type;
Tg: transgenic;
CHDs: congenital heart defects;
^{a}includes 5 ventricular septum defect (VSD), 1 hypoplastic left heart syndrome (HLHS), and 1 transposition of the great arteries (TGA);
^{b}includes 1 VSD;
*indicates significant differences (P < 0.05) compared with other groups by chi-square test.

TABLE 9
Mfn1 overexpression blocks CHD formation induced by
transgenic overexpression of miR-195 and miR-140

| | | Total | |
|---|---|---|---|
| Experimental group | Genotype | embryos | CHDs |
| Nkx2.5-miR-195- | WT | 23 | 0 |
| miR140 -Tg ♂ × | Mfn1 | 23 | 0 |
| Mfn1-Tg♀(n = 13) | 195-140 | 27 | 5 (18.5%)* |
| | Mfn1/195-140 | 24 | 0 |

WT: wildtype;
Tg: transgenic;
195-140: Nkx2.5-miR-195-miR140 -Tg;
Mfn1: Mfn1-Tg;
CHDs: congenital heart defects;
*indicates significant differences (P < 0.05) compared with other groups by chi-square test.

Similarly, Tg overexpression of Mfn2 also significantly reduced the incidence of CHDs in diabetic pregnancy (FIG. 10; Table 10), suggesting that Mfn1 and Mfn2 are redundant for mitochondrial fusion during cardiac development.

TABLE 10
Overexpression of Mfn2 in the embryonic heart
reduces CHD incidences in diabetic pregnancy

| | | Glucose | | Total | |
|---|---|---|---|---|---|
| Experimental group | | level(mg/dl) | Genotype | embryos | CHDs |
| ND | Mfn2-Tg ♂ × WT ♀ | 132.5 ± 9.4 | WT | 20 | 0 |
| | (n = 5) | | Tg | 21 | 0 |
| DM | Mfn2-Tg♂ × WT ♀ | 412.6 ± 24.7 | WT | 28 | 8^{a} (28.6%)* |
| | (n = 10) | | Tg | 29 | 1^{b} (3.45%) |

ND: nondiabetic;
DM: diabetic mellitus;
WT: wild type;
Tg: transgenic;
CHDs: congenital heart defects;
^{a}includes 7 ventricular septum defect (VSD) and 1 hypoplastic left heart syndrome (HLHS);
^{b}includes 1 VSD;
*indicates significant differences (P < 0.05) compared with other groups by chi-square test.

TABLE 11
The sequences of primers used in RT-qPCR.

| Primer Name | Primer Sequences (5′-3′) |
|---|---|
| miR-140 | F: CAGTGGTTTTACCCTATGGTAG |
| | (SEQ ID NO: 1) |
| miR-195 | F: TAGCAGCACAGAAATATTGGC |
| | (SEQ ID NO: 2) |
| Universal | From Mir-X miRNA First- |
| primer-R | Strand Synthesis Kit |
| | (Takara) |
| pri-miR-140 | F: TGGTGTGTGGTTCTATGCCAGC |
| | (SEQ ID NO: 3) |
| | R: AGCCTCAAGCCAGAATTCAGG |
| | (SEQ ID NO: 4) |
| pri-miR-195 | F: AAATATTGGCACAGGGAAGC |
| | (SEQ ID NO: 5) |
| | R: AGCCCCTCCTCGGTAGTTT |
| | (SEQ ID NO: 6) |
| Fox03a | F: CTGGGGGAACCTGTCCTATG |
| | (SEQ ID NO: 7) |
| | R: TCATTCTGAACGCGCATGAAG |
| | (SEQ ID NO: 8) |
| U6 | F: GTGCTCGCTTCGGCAGCACATAT |
| | (SEQ ID NO: 9) |
| | R: AAAAATATGGAACGCTTCACGAA |
| | (SEQ ID NO: 10) |
| Mfn1 | F: CCTTGTACATCGATTCCTGGGTT |
| | (SEQ ID NO: 11) |
| | R: CCTGGGCTGCATTATCTGGTG |
| | (SEQ ID NO: 12) |
| Mfn2 | F: AGAACTGGACCCGGTTACCA |
| | (SEQ ID NO: 13) |
| | R: CACTTCGCTGATACCCCTGA |
| | (SEQ ID NO: 14) |
| β-actin | F: GTGACGTTGACATCCGTAAAGA |
| | (SEQ ID NO: 15) |
| | R: GCCGGACTCATCGTACTCC |
| | (SEQ ID NO: 16) |

F: forward; R: reverse.

TABLE 12
The list of antibodies

| Antibody Name | Antibody Sources |
|---|---|
| Myomesin | Santa Cruz Biotechnology, Dallas, TX, |
| | Cat# sc-515638 |
| FoxO3a | Invitrogen, Carlsbad, CA, Cat # PA5-27145 |
| Ph3 | Cell Signaling Technology, Danvers, MA, Cat# 9701S |
| Mfn1 | Abcam, Cambridge, MA, Cat# ab221661 |
| Mfn2 | Cell Signaling Technology, Danvers, MA, Cat# 9482S |
| Drp1 | Abcam, Cambridge, MA, Cat# 118926 |
| Caspase 3 | Cell Signaling Technology, Danvers, MA, Cat# 9662S |
| AGO2 | Abcam, Cambridge, MA, Cat# ab186733 |
| β-actin | Abcam, Cambridge, MA, Cat# ab8224 |
| HRP-conjugated | Jackson ImmunoResearch Laboratories, West |
| goat anti-rabbit | Grove, PA, Cat#AP187 |
| HRP-conjugated | Jackson ImmunoResearch Laboratories, West |
| goat anti-mouse | Grove, PA, Cat#AP130 |
| Alexa-Fluor 488 | Invitrogen, Carlsbad, CA, Cat # A21206 |
| donkey | |
| anti-rabbit | |
| Alexa-Fluor 594 | Invitrogen, Carlsbad, CA, Cat # A21203 |
| donkey | |
| anti-mouse | |

Together, these results demonstrate that reduced mitochondrial fusion is a key event in the formation of CHDs induced by either miR-195/miR-140 Tg expression or exposure to a maternal diabetic milieu. Maternal diabetes-activated FoxO3a increases miR-140 and miR-195, which in turn represses Mfn1 and Mfn2, leading to mitochondrial fusion defects and CHDs. Maternal treatment with either of teriflunomide and echinacoside restores Mfn1 and Mfn2 expression and mitochondrial fusion in cardiomyocytes of embryonic hearts exposed to diabetes, implicating that activating mitochondrial fusion could be a potent means to prevent CHDs induced by maternal non-genetic factors.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1 Mathews, T. J. & Driscoll, A. K. Trends in Infant Mortality in the United States, 2005-2014. NCHS Data Brief, 1-8 (2017).
2. Virani S S, Alonso A, Benjamin E J et al. Heart Disease and Stroke Statistics-2020 Update: A Report From the American Heart Association. Circulation 2020; 141:e139-e596.
3. Botto L D, Mulinare J, Erickson J D. Occurrence of congenital heart defects in relation to maternal multivitamin use. Am J Epidemiol 2000; 151:878-84.
4. Oyen N, Olsen S F, Basit S et al. Association Between Maternal Folic Acid Supplementation and Congenital Heart Defects in Offspring in Birth Cohorts From Denmark and Norway. J Am Heart Assoc 2019; 8:e011615.
5. Jenkins K J, Correa A, Feinstein J A et al. Noninherited risk factors and congenital cardiovascular defects: current knowledge: a scientific statement from the American Heart Association Council on Cardiovascular Disease in the Young: endorsed by the American Academy of Pediatrics. Circulation 2007; 115:2995-3014.
6. Oyen N, Diaz L J, Leirgul E et al. Prepregnancy Diabetes and Offspring Risk of Congenital Heart Disease: A Nationwide Cohort Study. Circulation 2016; 133:2243-53.
7. Yang J, Cummings E A, O'Connell C, Jangaard K. Fetal and neonatal outcomes of diabetic pregnancies. Obstet Gynecol 2006; 108:644-50.
8. Loffredo C A, Wilson P D, Ferencz C. Maternal diabetes: an independent risk factor for major cardiovascular malformations with increased mortality of affected infants. Teratology 2001; 64:98-106.
9. Lawrence J M, Contreras R, Chen W, Sacks D A. Trends in the prevalence of preexisting diabetes and gestational diabetes mellitus among a racially/ethnically diverse population of pregnant women, 1999-2005. Diabetes Care 2008; 31:899-904.
10. Correa A, Gilboa S M, Besser L M et al. Diabetes mellitus and birth defects. Am J Obstet Gynecol 2008; 199:237 e1-9.
11. Xu C, Shen W B, Reece E A et al. Maternal diabetes induces senescence and neural tube defects sensitive to the senomorphic rapamycin. Sci Adv 2021; 7.
12. Yang P, Xu C, Reece E A et al. Tip60- and sirtuin 2-regulated MARCKS acetylation and phosphorylation are required for diabetic embryopathy. Nat Commun 2019; 10:282.
13. Wang F, Xu C, Reece E A et al. Protein kinase C-alpha suppresses autophagy and induces neural tube defects via miR-129-2 in diabetic pregnancy. Nat Commun 2017; 8:15182.
14. Li J, Li Y, Jiao J et al. Mitofusin 1 is negatively regulated by microRNA 140 in cardiomyocyte apoptosis. Mol Cell Biol 2014; 34:1788-99.
15. Ong S B, Hall A R, Hausenloy D J. Mitochondrial dynamics in cardiovascular health and disease. Antioxid Redox Signal 2013; 19:400-14.
16. Kasahara A, Cipolat S, Chen Y, Dorn G W, 2nd, Scorrano L. Mitochondrial fusion directs cardiomyocyte differentiation via calcineurin and Notch signaling. Science 2013; 342:734-7.
17. Wang F, Fisher S A, Zhong J, Wu Y, Yang P. Superoxide Dismutase 1 In Vivo Ameliorates Maternal Diabetes Mellitus-Induced Apoptosis and Heart Defects Through Restoration of Impaired Wnt Signaling. Circ Cardiovasc Genet 2015; 8:665-76.
18. Chen H, Detmer S A, Ewald A J, Griffin E E, Fraser S E, Chan D C. Mitofusins Mfn1 and Mfn2 coordinately regulate mitochondrial fusion and are essential for embryonic development. J Cell Biol 2003; 160:189-200.
19. Karbowski M, Arnoult D, Chen H, Chan D C, Smith C L, Youle R J. Quantitation of mitochondrial dynamics by photolabeling of individual organelles shows that mitochondrial fusion is blocked during the Bax activation phase of apoptosis. J Cell Biol 2004; 164:493-9.
20. Miret-Casals L, Sebastián D, Brea J et al. Identification of New Activators of Mitochondrial Fusion Reveals a Link between Mitochondrial Morphology and Pyrimidine Metabolism. Cell Chem Biol 2018; 25:268-278.e4.
21. Zeng K W, Wang J K, Wang L C et al. Small molecule induces mitochondrial fusion for neuroprotection via targeting CK2 without affecting its conventional kinase activity. Signal Transduct Target Ther 2021; 6:71.
22. Zhang G Q, Wang S Q, Chen Y et al. MicroRNAs regulating mitochondrial function in cardiac diseases. Front Pharmacol 2021; 12:663322.
23. Yang P, Li X, Xu C et al. Maternal hyperglycemia activates an ASK1-FoxO3a^{-/-} caspase 8 pathway that leads to embryonic neural tube defects. Sci Signal 2013; 6:ra74.
24 Wang F, Wu Y, Quon M J, Li X, Yang P. ASK1 mediates the teratogenicity of diabetes in the developing heart by inducing E R stress and inhibiting critical factors essential for cardiac development. Am J Physiol Endocrinol Metab 2015; 309:E487-99.
25. Kitani T, Tian L, Zhang T et al. RNA sequencing analysis of induced pluripotent stem cell-derived cardiomyocytes from congenital heart disease patients. Circ Res 2020; 126:923-925.
26. Ouyang Z, Wei K. miRNA in cardiac development and regeneration. Cell Regen 2021; 10:14.
27. Zhao Y, Samal E, Srivastava D. Serum response factor regulates a muscle-specific microRNA that targets Hand2 during cardiogenesis. Nature 2005; 436:214-20.
28. Liu N, Bezprozvannaya S, Williams A H et al. microRNA-133a regulates cardiomyocyte proliferation and suppresses smooth muscle gene expression in the heart. Genes Dev 2008; 22:3242-54.
29. Tuddenham L, Wheeler G, Ntounia-Fousara S et al. The cartilage specific microRNA-140 targets histone deacetylase 4 in mouse cells. FEBS Lett 2006; 580:4214-7.
30. Zhou J, Dong X, Zhou Q et al. microRNA expression profiling of heart tissue during fetal development. Int J Mol Med 2014; 33:1250-60.
31. Okada M, Kim H W, Matsu-ura K, Wang Y G, Xu M, Ashraf M. Abrogation of age-induced microRNA-195 rejuvenates the senescent mesenchymal stem cells by reactivating telomerase. Stem Cells 2016; 34:148-59.
32. van Rooij E, Sutherland L B, Liu N et al. A signature pattern of stress-responsive microRNAs that can evoke cardiac hypertrophy and heart failure. Proc Natl Acad Sci USA 2006; 103:18255-60.
33. Divakaran V, Mann D L. The emerging role of microRNAs in cardiac remodeling and heart failure. Circ Res 2008; 103:1072-83.
34. Song Y, Zeng S, Zheng G et al. FOXO3a-driven miRNA signatures suppresses VEGF-A/NRP1 signaling and breast cancer metastasis. Oncogene 2021; 40:777-790.
35. Skurk C, Izumiya Y, Maatz H et al. The FOXO3a transcription factor regulates cardiac myocyte size downstream of AKT signaling. J Biol Chem 2005; 280:20814-23.
36. Chaanine A H, Jeong D, Liang L et al. JNK modulates FOXO3a for the expression of the mitochondrial death and mitophagy marker BNIP3 in pathological hypertrophy and in heart failure. Cell Death Dis 2012; 3:265.
37. Li X, Du N, Zhang Q et al. MicroRNA-30d regulates cardiomyocyte pyroptosis by directly targeting foxo3a in diabetic cardiomyopathy. Cell Death Dis 2014; 5:e1479.
38. Yang P, Yang W W, Chen X, Kaushal S, Dong D, Shen W B. Maternal diabetes and high glucose in vitro trigger Sca1(+) cardiac progenitor cell apoptosis through FoxO3a. Biochem Biophys Res Commun 2017; 482:575-581.
39. Detmer S A, Chan D C. Functions and dysfunctions of mitochondrial dynamics. Nat Rev Mol Cell Biol 2007; 8:870-9.
40. Chen H, McCaffery J M, Chan D C. Mitochondrial fusion protects against neurodegeneration in the cerebellum. Cell 2007; 130:548-62.
41. Mitra K, Wunder C, Roysam B, Lin G, Lippincott-Schwartz J. A hyperfused mitochondrial state achieved at G1-S regulates cyclin E buildup and entry into S phase. Proc Natl Acad Sci USA 2009; 106:11960-5.
42. Chen H, Chomyn A, Chan D C. Disruption of fusion results in mitochondrial heterogeneity and dysfunction. J Biol Chem 2005; 280:26185-92.
43. Duvezin-Caubet S, Jagasia R, Wagener J et al. Proteolytic processing of OPA1 links mitochondrial dysfunction to alterations in mitochondrial morphology. J Biol Chem 2006; 281:37972-9.
44. Liu J, Yang L, Dong Y, Zhang B, Ma X. Echinacoside, an Inestimable Natural Product in Treatment of Neurological and other Disorders. 2018; 23.
45. Deng M, Zhao J Y, Tu P F, Jiang Y, Li Z B, Wang Y H. Echinacoside rescues the SHSY5Y neuronal cells from TNFalpha-induced apoptosis. Eur J Pharmacol 2004; 505:11-8.
46. Castrillon D H, Miao L, Kollipara R, Horner J W, DePinho R A. Suppression of ovarian follicle activation in mice by the transcription factor FoxO3a. Science 2003; 301:215-8.
47. Miyaki S, Sato T, Inoue A et al. MicroRNA-140 plays dual roles in both cartilage development and homeostasis. Genes Dev 2010; 24:1173-85.
48. Rönicke V, Risau W, Breier G. Characterization of the endothelium-specific murine vascular endothelial growth factor receptor-2 (Flk-1) promoter. Circ Res 1996; 79:277-85.
49. Li X, Xu C, Yang P. c-Jun NH2-terminal kinase 1/2 and endoplasmic reticulum stress as interdependent and reciprocal causation in diabetic embryopathy. Diabetes 2013; 62:599-608.
50. Karbowski M, Cleland M M, Roelofs B A. Photoactivatable green fluorescent protein-based visualization and quantification of mitochondrial fusion and mitochondrial network complexity in living cells. Methods Enzymol 2014; 547:57-73.
51. Li S, Xu S, Roelofs B A et al. Transient assembly of F-actin on the outer mitochondrial membrane contributes to mitochondrial fission. J Cell Biol 2015; 208:109-23.
52. Zhong J, Reece E A, Yang P. Punicalagin exerts protective effect against high glucose-induced cellular stress and neural tube defects. Biochem Biophys Res Commun 2015; 467:179-84.
53. Wang G, Song S, Shen W B, Reece E A, Yang P. MicroRNA-322 overexpression reduces neural tube defects in diabetic pregnancies. Am J Obstet Gynecol 2023.
54. Gu H, Yu J, Dong D, Zhou Q, Wang J Y, Yang P. The miR-322-TRAF3 circuit mediates the pro-apoptotic effect of high glucose on neural stem cells. Toxicol Sci 2015; 144:186-96.
55. Tran H, Brunet A, Grenier J M et al. DNA repair pathway stimulated by the forkhead transcription factor FOXO3a through the Gadd45 protein. Science 2002; 296:530-4.

What is claimed is:

1. A method of preventing congenital heart defects (CHDs) in an embryo of a pregnant diabetic female subject, said method comprising administering to a pregnant diabetic female subject and/or an embryo of a pregnant diabetic female subject a therapeutically-effective amount of at least one mitochondrial fusion activator.

2. A method of restoring or augmenting mitochondrial fusion in an embryo of a pregnant diabetic female subject, said method comprising administering to a pregnant diabetic female subject and/or an embryo of a pregnant diabetic female subject a therapeutically-effective amount of at least one mitochondrial fusion activator.

3. A method of inducing or augmenting expression of mitofusin 1 (Mfn1) and/or mitofusin 2 (Mfn2) in an embryo of a pregnant diabetic female subject, said method comprising administering to a pregnant diabetic female subject and/or an embryo of a pregnant diabetic female subject a therapeutically-effective amount of at least one mitochondrial fusion activator.

4. The method of claim 2, wherein mitochondrial fusion is restored or augmented in cardiomyocytes.

5. The method of claim 3, wherein Mfn1 and/or Mfn2 expression is induced or augmented in cardiomyocytes.

6. The method of claim 1, wherein the mitochondrial fusion activator is teriflunomide (TERI) or echinacoside (ECH), or both TERI and ECH.

7. The method of claim 2, wherein the mitochondrial fusion activator is teriflunomide (TERI) or echinacoside (ECH), or both TERI and ECH.

8. The method of claim 3, wherein the mitochondrial fusion activator is teriflunomide (TERI) or echinacoside (ECH), or both TERI and ECH.

9. The method of claim 6, wherein the therapeutically-effective amount of TERI is between 1 mg/kg and 30 mg/kg.

10. The method of claim 6, wherein the therapeutically-effective amount of ECH is between 1 mg/kg and 30 mg/kg.

11. The method of claim 1, wherein the embryo is in the first or early second trimester.

12. The method of claim 2, wherein the embryo is in the first or early second trimester.

13. The method of claim 3, wherein the embryo is in the first or early second trimester.

14. The method of claim 1, wherein the mitochondrial fusion activator is administered to the amniotic cavity of the embryo.

15. The method of claim 2, wherein the mitochondrial fusion activator is administered to the amniotic cavity of the embryo.

16. The method of claim 3, wherein the mitochondrial fusion activator is administered to the amniotic cavity of the embryo.

17. The method of claim 14, wherein the administration is via a microinjection.

18. The method of claim 1, wherein the female subject has gestational diabetes.

19. The method of claim 2, wherein the female subject has gestational diabetes.

20. The method of claim 3, wherein the female subject has gestational diabetes.
